(19) United States
(12) Patent Application Publication
(10) Pub. No.: US 2023/0241152 A1
(43) Pub. Date: Aug. 3, 2023

(54) SYNERGISTIC HERBAL COMPOSITIONS FOR IMPROVING GUT HEALTH

(71) Applicant: LAILA NUTRACEUTICALS, Vijayawada (IN)

(72) Inventors: Ganga Raju GOKARAJU, Vijayawada (IN); Rama Raju Gokaraju, Vijayawada (IN); Kiran BHUPATHIRAJU, Vijayawada (IN); Venkata Kanaka Ranga Raju GOKARAJU, Vijayawada (IN); Trimurtulu GOLAKOTI, Vijayawada (IN); Krishanu SENGUPTHA, Vijayawada (IN); Venkateswarlu SOMEPALLI, Vijayawada (IN); Venkata Krishna Raju ALLURI, Vijayawada (IN)

(21) Appl. No.: 18/000,146

(22) PCT Filed: May 29, 2021
(86) PCT No.: PCT/IN2021/050526

§ 371 Date: Nov. 29, 2022

(30) Foreign Application Priority Data
May 29, 2020 (IN) 202041022680

(51) Int. Cl. A61K 36/888; A23L 33/00; A23L 33/105; A61K 36/185; A61K 36/81; A61P 1/14

(52) U.S. Cl. CPC A61K 36/888; A23L 33/40; A23L 33/105; A61K 36/185; A61K 36/81; A61P 1/14; A61K 2236/331; A61K 2236/333

(57) ABSTRACT

Synergistic Herbal Compositions For Improving Gut Health” The present invention discloses synergistic herbal compositions comprising at least two ingredients selected from extract(s), fraction(s), phytochemical(s), or mixtures thereof derived each one from a different herb selected from Abelmoschus esculentus, Withania somnifera and Amorphophallus paeoniifolius; improving at least one gut health function selected from gut-brain axis, easing of constipation, bowel movement, improved GI motility, digestion and gut immunity; and reducing gut inflammation. The invention further relates to methods of treatment and the use of the compositions.

### TECHNICAL FIELD OF THE INVENTION

The invention relates to synergistic herbal compositions comprising at least two ingredients selected from extract(s), fraction(s), phytochemical(s), or mixtures thereof derived each one from a different herb selected from Abelmoschus esculentus, Withania somnifera and Amorphophallus paeoniifolius; for improving at least one gut health function selected from gut-brain axis, easing of constipation, bowel movement, gastrointestinal (GI) motility, digestion and gut immunity; and reducing gut inflammation.

### BACKGROUND OF THE INVENTION

The gut is one of the most important and complex organ in most living systems. It plays multiple roles as a digestive, immune, and endocrine organ; besides it also possesses a nervous system that is called an enteric nervous system (ENS). Hence, it is widely referred to as a second brain. The autonomic nervous system (ANS), hypothalamic-pituitary-adrenal (HPA) axis, and the nerves within the gastrointestinal (GI) tissues, all link the gut and the brain, allowing the mind to influence intestinal activities, including activity of functional immune effector cells; and similarly allowing the gut to modulate mood, cognition, mental health and maintains homeostasis through brain-gut-microbiota axis. Common gastrointestinal functions such as digestion, bowel movement, immunological homeostasis are closely linked to psychological status. Recent research has elucidated four major pathways involved in the gut-brain interaction: neurologic, endocrine, humoral/metabolic, and immune. The neurological pathway includes the vagus nerve, the ENS, and the neurotransmitters in the GI tissue. Neurologic modulation of afferent sensory nerves produces neurotransmitters such as GABA, serotonin, melatonin, histamine, and acetylcholine, along with biologically active forms of catecholamines in the lumen of the gut. The endocrine pathway involves biologically active peptides from enteroendocrine cells such as galanin, which stimulates the HPA axis and enhances glucocorticoid secretion from the adrenal cortex. The humoral/metabolic pathway involves gut bacterial metabolites best known to affect the nutrition of enterocytes and possess hormone-like activity, immunomodulatory properties. They interact with nerve cells by stimulating the sympathetic branch of the ANS.

Furthermore, microbiota-derived short-chain fatty acids (SCFAs) cross the blood-brain barrier and regulate microglia homeostasis involved in behavior modulation. The immune pathway involves inflammation and metabolism within the GI tract, influences by the gut microbiome, via the release of cytokines like IL-4, IFN-γ, and IL-10 during the times of alteration of gut microbiota called dysbiosis. Disturbances in the brain-gut-microbiota axis result in digestive disorders, impaired gut motility, and constipation. Several factors, including stress, mood disorders like anxiety and depression are linked with functional GI disturbances. Overall, the gut plays a vital role in maintaining general health and well-being.

Constipation is generally defined as an infrequent and difficult passage of stool. It is estimated that approximately one in every 50 people in the USA suffers from constipation, making it one of the common disorders among Americans. It is more likely to affect females than males and more likely to occur in older adults, with a steep increase after 65 years. The actual estimates are likely higher than reported, as many individuals suffer at home without seeking medical help.

Synthetic laxative agents tend to cause some side effects such as rectal bleeding, bloody stools, severe crams or pain, weakness or unusual tiredness, dizziness, etc.

Patent application US2015037315A1 disclosed a composition of kiwifruit extract in combination with an amylase inhibitor and/or a bifunctional protease-amylase inhibitor, particularly, but not exclusively, for managing gut health or for the treatment or prevention of digestive dysfunction, gastrointestinal disorders, or symptoms thereof.

Another patent application CA2981834A1 disclosed the use of plant extract blends, comprising: i. Grape seed extract rich in polyphenols and ii. Apple fruit extract rich in polyphenols and iii. Cranberry fruit extract rich in polyphenols and IV. Black currant fruit extract rich in polyphenols and v. Resveratrol rich in polyphenols for improving gut health and/or improving metabolism.

Another patent publication WO2018015353A1 disclosed a composition with a laxative effect which comprises a tamarind (Tamarindus indica L.) extract, a ginger (Zingiber officinale Rose.) extract, and a buckthorn (Frangula dodonei) extract, wherein the buckthorn extract is in a weight ratio less than or equal to 0.03 with respect to the tamarind extract.

Another patent publication WO2019091812A1 disclosed a composition containing a synergistic association of the extract of at least one plant belonging to the genus Plantago, glucomannan, and at least an extract of chamomile for the acute and chronic treatment of constipation.

Another patent publication U.S. Ser. No. 10/449,225B1 disclosed a method for restoring stability to the function of the human gastrointestinal tract by providing relief from any symptom selected from the group consisted of constipation, diarrhea, and gastroesophageal reflux disease; said method comprising orally administering to a human subject who is suffering from said symptom a composition consisting essentially of an extract of the raw fruits of Coffee arabica, said extract being present in said composition in any amount between 50% by weight and 99% by weight, based on the total weight of the said composition.

Thus, there is a need in the art for better treatment options with minimal side effects thereby making the option safe for human consumption, especially when used in long term therapy.

### OBJECTIVE OF THE INVENTION

Therefore, the main object of the present invention is to provide synergistic and safe herbal compositions comprising at least two ingredients selected from extract(s), fraction(s), phytochemical(s), or mixtures thereof derived each one from a different herb selected from Abelmoschus esculentus, Withania somnifera and Amorphophallus paeoniifolius; for improving at least one gut health function selected from gut-brain axis, easing of constipation, bowel movement, GI motility, digestion and gut immunity; and reducing gut inflammation.

Another objective of the invention is to provide methods of improving at least one gut health function selected from gut-brain axis, easing of constipation, bowel movement, GI motility, digestion and gut immunity; and reducing gut inflammation; in a human, wherein the method comprises supplementing human with an effective dose of a composition comprising at least two ingredients selected from extract(s), fraction(s), phytochemical(s), or mixtures thereof derived each one from a different herb selected from Abelmoschus esculentus, Withania somnifera and Amorphophallus paeoniifolius and optionally containing at least one component selected from pharmaceutically, nutraceutically or dietically acceptable excipients, carriers and diluents.

Yet another objective of the invention is to provide the use of synergistic compositions comprising at least two ingredients selected from extract(s), fraction(s), phytochemical(s), or mixtures thereof derived each one from a different herb selected from Abelmoschus esculentus, Withania somnifera and Amorphophallus paeoniifolius and optionally containing at least one component selected from pharmaceutically, nutraceutically or dietically acceptable excipients, carriers and diluents; for improving at least one gut health function selected from gut-brain axis, easing of constipation, bowel movement, GI motility, digestion and gut immunity; and reducing gut inflammation.

### SUMMARY OF THE INVENTION

The present invention provides synergistic herbal compositions comprising at least two ingredients selected from extract(s), fraction(s), phytochemical(s), or mixtures thereof derived each one from a different herb selected from Abelmoschus esculentus, Withania somnifera and Amorphophallus paeoniifolius; for improving at least one gut health function selected from gut-brain axis, easing of constipation, bowel movement, GI motility, digestion and gut immunity; and reducing gut inflammation.

Another aspect of the invention provides synergistic herbal compositions comprising at least two ingredients selected from extract(s), fraction(s), phytochemical(s), or mixtures thereof derived each one from a different herb selected from Abelmoschus esculentus, Withania somnifera and Amorphophallus paeoniifolius and optionally containing at least one component selected from pharmaceutically or nutraceutically or dietically acceptable excipients, carriers and diluents.

Other aspect of the invention provides methods of improving at least one gut health function selected from gut-brain axis, easing of constipation, bowel movement, GI motility, digestion and gut immunity; and reducing gut inflammation; in a human, wherein the method comprises supplementing the human with an effective dose of a composition comprising at least two ingredients selected from extract(s), fraction(s), phytochemical(s), or mixtures thereof derived each one from a different herb selected from Abelmoschus esculentus, Withania somnifera and Amorphophallus paeoniifolius and optionally containing at least one component selected from pharmaceutically or nutraceutically or dietetically acceptable excipients, carriers and diluents.

Another aspect of the invention provides the use of synergistic compositions comprising at least two ingredients selected from extract(s), fraction(s), phytochemical(s), or mixtures thereof derived each one from a different herb selected from Abelmoschus esculentus, Withania somnifera and Amorphophallus paeoniifolius and optionally containing at least one component selected from pharmaceutically, nutraceutically or dietetically acceptable excipients, carriers and diluents; for improving at least one gut health function selected from gut-brain axis, easing of constipation, bowel movement, GI motility, digestion and gut immunity; and reducing gut inflammation.

### DESCRIPTION OF FIGURES

FIG. 1. Bar diagrams depict the improvement of fecal pellet count (A), fecal moisture content (B), intestinal transit (C), and gastric emptying (D) in experimental Wistar rats supplemented individual ingredients and their compositions. The groups represent Abelmoschus esculentus water extract (A.E-1, 300 mg/Kg), a blend of Withania somnifera water and spent 80% aqueous ethanol extracts (W.S-2, 300 mg/Kg), Withania somnifera water extract (W.S-1, 300 mg/Kg), composition-8 comprising Abelmoschus esculentus water extract (A.E-1) and a blend of Withania somnifera water and spent 80% aqueous ethanol extracts (W.S-2) in 1:1 ratio (300 mg/Kg) and composition-3 comprising Abelmoschus esculentus water extract (A.E-1) and Withania somnifera water extract (W.S-1) in 1:1 ratio (300 mg/Kg) respectively. Each bar presents the mean±SD; n=6. The figures in parentheses (above the bars) show the changes (in percentage) from vehicle control (G1). * indicates significance (p<0.05), one-way ANOVA, vs. Vehicle control. FIG. 1A represents fecal pellet count and the values on the top of the bars are % improvement from day 1; FIG. 1B represents fecal moisture content and the values on the top of the bars are % improvement from day 1; FIG. 1C represents intestinal transit and the values on the top of the bars are % increase from normal control; FIG. 1D represents gastric emptying and the values on the top of the bars are % increase from normal control.

### DESCRIPTION OF THE INVENTION

The invention will now be described in detail in connection with certain preferred and optional embodiments so that various aspects thereof may be more fully understood and appreciated.

To address the problem and to provide a safe herbal composition(s) for improving gut health, the present inventors have aimed at providing synergistic compositions comprising at least two ingredients selected from extract(s), fraction(s), phytochemical(s), or mixtures thereof derived each one from a different herb selected from Abelmoschus esculentus, Withania somnifera and Amorphophallus paeoniifolius for relieving the symptoms of constipation through improved gastrointestinal motility, increased amount of stool, and its softness (i.e., reduced dryness). To achieve these objectives, the following experiments were chosen.
1. An ex-vivo (isolated tissue) experiment conducted on the isolated ileum of male Wistar rats to evaluate the smooth muscle contractility.
2. An in-vivo study in male Wistar rats for evaluating the gastrokinetic activity (gastrointestinal movement), amount of fecal matter and its wetness and the influence on the key neurohormones regulating the GI activities.

Constipation is infrequent bowel movements or difficult passage of stools that persists for several weeks or longer. It is associated with the passage of small amounts of hard stool due to dry and sluggish bowel movements, usually fewer than three times a week. People with constipation may find it difficult and painful to have a bowel clearance. Other symptoms of constipation include feeling bloated, uncomfortable, and sluggish. During the digestion process food moves through the colon, and it absorbs excess water while forming waste products or stool. The periodic muscle contraction-relaxation cycle (peristalsis) generates force to push the stool towards the rectum. The frequency of the contraction-relaxation cycle and its force are regulated by the peristaltic wave generated from the upper part of the GI tract, i.e., the distal part of the small intestine or ileum. However, by the time the stool reaches the rectum, it becomes solid because most of the water has been absorbed. In constipation, the colon's muscle contractions are slow or sluggish, causing the stool to stay longer in the colon, resulting in hard and dry stool, which is too difficult to pass. Therefore, an ex-vivo experimental model was chosen to evaluate the herbal extracts and their compositions for their efficacy to increase the contractions of the gastrointestinal tissues in isolated ileum of male Sprague Dawley rats. Further, an in-vivo efficacy experiment was conducted in male Sprague Dawley rats to evaluate whether the inventive synergistic herbal composition could provide relief from the symptoms of constipation. All relevant parameters such as the gastrointestinal motility (gastro kinetic activity), amount of feces and its wetness, and the key neurohormones responsible for GI movements were assessed to evaluate the impact of the composition on the gut-brain cross-talk (gut-brain axis).

Source of the herbs used in the invention as follows:
1) Abelmoschus esculentus fruit raw material was collected from Kankipadu village, Krishna district, Andhra Pradesh and it was cultivated.
2) Withania somnifera root raw material was obtained from Siddarampuram village, Anantapur district, Andhra Pradesh and it was cultivated.
3) Amorphophallus paeoniifolius tuber raw material was collected from Kunchanapalli village, Guntur district, Andhra Pradesh and it was cultivated.

A brief summary of each of the plant material is provided herein below.

Abelmoschus esculentus, most commonly known as okra or ladies finger, is cultivated as an important vegetable crop in tropical, subtropical, and warm temperate regions worldwide. It is annual or perennial; plant belongs to the family Malvaceae. Okra seeds are the source of oil and protein. It can be used as a non-caffeinated substitute for coffee. In Ayurveda, okra is used as an edible infusion and in different preparations for diuretic effect. An infusion of the fruit mucilage is also used to treat dysentery and diarrhea in acute inflammation and irritation of the stomach, bowels, and kidneys catarrhal infections, dysuria, and gonorrhea. Okra pod contains important bioactive compounds such as carotene, folic acid, thiamine, riboflavin, niacin, vitamin C, oxalic acid, amino acids, and polysaccharides. Besides, this plant is also the treasure house of polyphenolic compounds such as hyperoside, quercetin, coumarin scopoletin, uridine, and phenylalanine.

Pharmacologically the okra fruit is used for gastroprotective, anti-adhesive effect, antioxidant, anti-stress, nootropic activities, hypolipidemic, anti-diabetic, eye sight improvement and skin nourishment activities (Anupam Roy et al., Plant Science Today, 2014, 1(3), 121-130; Rakesh K Sindhu et al., The Journal of Phytopharmacology, 2016, 5(6), 238-241).

Withania somnifera is a small evergreen shrub that grows roughly four to five feet tall and belongs to Solanaceae family, most commonly known as Ashwagandha or winter cherry. It is the most valuable plant in traditional Indian systems of medicine. Herbalists refer to Ashwagandha as Indian ginseng since its uses in Ayurvedic medicine are similar to those of ginseng in Traditional Chinese Medicine (TCM). The phytochemical constituents of Withania somnifera roots are withanolides and withanolide glycosides. Ashwagandha has been reported with various pharmacological activities such as analgesic, hypoglycemic, diuretic, and hypocholesterolemic, adjuvant to chemotherapy; for cardiovascular protection, erectile dysfunction, anti-arthritic, growth-promoting, rejuvenating, infertility, impotence, repeated miscarriage, paralysis, memory loss, etc. (Krutika Joshi et al., International Journal of Pharmaceutical & Biological Archives, 2016, 7(1), 1-11; Veena Sharma et al., International Journal of Pharm Tech Research, 2011, 3(1), 187-192).

Amorphophallus paeoniifolius (Syn Amorphophallus campanulatus or elephant foot yam) of the family Areaceae is a tuberous plant commonly used in Ayurvedic medicines as well as tribal medicines in India. It is a stout herbaceous plant with an underground hemispherical depressed dark brown corm plant, which is cultivated largely throughout India and other parts of the world. Tuber is a delicacy in food and rich in nutrients. It is very popular as a vegetable in various delicious cuisines. It is acrid, astringent, thermogenic, and traditionally used in vitiated condition of vata and kapha, arthralgia, elephantiasis, tumors, inflammations, haemorrhoids, haemorrhages, vomiting, cough, bronchitis, asthma, anorexia, dyspepsia, flatulence, colic, constipation, helminthiasis hepatopathy, splenopathy, amenorrhoea, dysmenorrhoea, seminal weakness, fatigue, anemia and general debility. Amorphophallus is a good source of energy, sugar, starch, proteins as well as minerals. Tubers contain alkaloids, steroids, flavonoids and carbohydrates, tannins, etc. The corm/tubers are gastroprotective, analgesic, anti-convulsant, antihelmintic, antidiarrhoeal, anti-inflammatory, hepatoprotective, CNS depressant etc. (Anuradha Singh et al., Int. J. Pharm. Sci. Rev. Res., 2014, 24(1), 55-60).

Abelmoschus esculentus fruit raw material was pulverized, and the powder was extracted with water to obtain water extract (A.E-1). To enrich polysaccharides, the water extract of Abelmoschus esculentus fruit was treated with ethanol to get ethanol precipitated water extract (A.E-2). The Abelmoschus esculentus fruit powder was extracted with other solvents such as 50% aqueous ethanol, 50% aqueous methanol, and 20% aqueous acetone to obtain 50% aqueous ethanol extract (A.E-3), 50% aqueous methanol extract (A.E-4), and 20% aqueous acetone extract (A.E-5) respectively. The said extracts of Abelmoschus esculentus fruit were standardized to total polysaccharides by UV method of analysis, and the results are summarized in Table 1.

Similarly, the Withania somnifera dried root was pulverized and the powder was extracted with water and the extract concentrated to obtain water extract (W.S-1). The spent raw material obtained after water wash was extracted again with 80% ethanol, and the dried extract was combined with water extract to get a composition of water & 80% aqueous ethanol extracts (W.S-2). The Withania somnifera dried root powder was also extracted with other solvents such as 80% aqueous methanol and 80% aqueous acetone to obtain 80% aqueous methanol extract (W.S-3) and 80% aqueous acetone extract (W.S-4), respectively. These extracts of Withania somnifera root were standardized to total withanolides (withanolides and withanolide glycosides) by analytical HPLC method of analysis and the results are summarized in Table 2.

Similarly, dried Amorphophallus paeoniifolius tuber was pulverized, and the powder was extracted with various solvents such as water, 50% aqueous ethanol, and ethanol to obtain water extract (A.P-1), 50% aqueous ethanol extract (A.P-2), and ethanol extract (A.P-3), respectively.

The inventors screend a number of plant extracts for smooth muscle contractility activity in isolated rat ileum (ex-vivo) and finally selected extracts of Abelmoschus esculentus, Withania somnifera, and Amorphophallus paeoniifolius for further development as they showed better efficacy. Then, various compositions were prepared by combining two ingredients selected from extract(s), fraction(s), phytochemical(s), and mixtures thereof at different ratios, where in each ingredient in the compositoin was derived from a different herb selected from Abelmoschus esculentus, Withania somnifera, and Amorphophallus paeoniifolius. The compositions so obtained were evaluated for smooth muscle contractility activity in isolated rat ileum (ex-vivo) in comparison with the corresponding individual ingredients. The EC50 (half-maximal effective concentration) data from ex-vivo study showed that these compositions unexpectedly have better efficacy in improving contractility on smooth muscle when compared to their corresponding individual ingredients suggesting that the compositions containing the extracts derived from Abelmoschus esculentus, Withania somnifera, and Amorphophallus paeoniifolius have the tendency to exhibit synergism.

For example, Abelmoschus esculentus water extract (A.E-1) and Withania somnifera water extract (W.S-1) showed efficacy in improving smooth muscle contractility with EC50 values of 24.62 μg/mL and 26.80 μg/mL, respectively. The composition-1 containing these two extracts at 3:1 ratio showed EC50 value of 16.00 μg/mL, which is significantly lower indicating a higher efficacy than the individual ingredients, suggesting a synergistic effect between Abelmoschus esculentus water extract (A.E-1) and Withania somnifera water extract (W.S-1) in improving contractility on smooth muscle (Lower the EC50 value is higher the efficacy). The compositions-3 and 5, which were obtained when combining these two ingredients at ratios, 1:1 and 1:3, respectively, also showed synergism in smooth muscle contractility, when compared to the efficacy demonstrated by their corresponding individual ingredients as summarized in Table-3.

In another example, Abelmoschus esculentus water extract (A.E-1) and Withania somnifera water & spent 80% aqueous ethanol blend extract (W.S-2) exhibited smooth muscle contractility with EC50 values of 24.62 μg/mL and 24.79 μg/mL, respectively. The composition-6 containing these two extracts at 3:1 ratio showed an EC50 value of 15.98 μg/mL, which is significantly lower indicating a higher efficacy than the individual ingredients This result suggests a synergistic effect between Abelmoschus esculentus water extract (A.E-1) and Withania somnifera water & 80% aqueous ethanol blend extract (W.S-2) in improving contractility efficacy on smooth muscle (Lower the EC50 value is higher the efficacy). The compositions-8 and -10 containing these two ingredients at ratios, 1:1 and 1:3 respectively, also showed synergism in improving smooth muscle contractility, when compared to the efficacy demonstrated by their corresponding individual ingredients as summarized in Table-4.

In another example, Abelmoschus esculentus ethanol precipitated water extract (A.E-2) and Withania somnifera water & 80% aqueous ethanol blend extract (W.S-2) exhibited smooth muscle contractility with EC50 values of 19.69 μg/mL and 22.31 μg/mL, respectively. The composition-11 containing these two extracts at 2:1 ratio showed an EC50 value of 14.97 μg/mL, which is lower indicating a higher efficacy the individual ingredients, suggesting a synergistic effect between Abelmoschus esculentus ethanol precipitated water extract (A.E-2) and Withania somnifera water & 80% aqueous ethanol blend extract (W.S-2) in improving contractility efficacy on smooth muscle. The composition-13 containing these two ingredients at a 1:2 ratio also showed synergism in improving smooth muscle contractility when compared to the efficacy shown by their corresponding individual ingredients as summarized in Table-5.

In another example, Abelmoschus esculentus 50% aq ethanol extract (A.E-3), and Withania somnifera water & 80% aqueous ethanol blend extract (W.S-2) exhibited smooth muscle contractility with EC50 values of 25.54 μg/mL and 21.33 μg/mL respectively. The composition-14 containing these two extracts at 2:1 ratio showed an EC50 value of 15.47 μg/mL, which is significantly higher than the individual ingredients, suggesting a synergistic effect between Abelmoschus esculentus 50% aq ethanol extract (A.E-3) and Withania somnifera water & 80% aqueous ethanol blend extract (W.S-2) in improving contractility efficacy on smooth muscle. The composition-16 obtained when combining these two ingredients at a 1:2 ratio also showed synergism in improving smooth muscle contractility when compared to the efficacy demonstrated by their corresponding individual ingredients as summarized in Table-6.

In another example, Abelmoschus esculentus 50% aq methanol extract (A.E-4) and Withania somnifera 80% aqueous acetone extract (W.S-4) exhibited smooth muscle contractility with EC50 values of 22.98 μg/mL and 21.71 μg/mL respectively. The composition-17 containing these two extracts at 2:1 ratio showed an EC50 value of 16.16 μg/mL, which is significantly higher than the individual ingredients, suggesting a synergistic effect between Abelmoschus esculentus 50% aq methanol extract (A.E-4) and Withania somnifera 80% aqueous acetone extract (W.S-4) in improving contractility efficacy on smooth muscle. The composition-19 obtained when combining these two ingredients at a 1:2 ratio also showed synergism in improving smooth muscle contractility when compared to the efficacy demonstrated by their corresponding individual ingredients as summarized in Table-7.

In another example, Abelmoschus esculentus 20% aq acetone extract (A.E-5), and Withania somnifera 80% aq methanol extract (W.S-3) exhibited smooth muscle contractility with EC50 values of 22.31 μg/mL and 22.03 μg/mL respectively. The composition-20 containing these two extracts at 2:1 ratio showed an EC50 value of 10.11 μg/mL, which is significantly better than the individual ingredients, suggesting synergistic effect between Abelmoschus esculentus 20% aq acetone extract (A.E-5) and Withania somnifera 80% aq methanol extract (W.S-3) in improving contractility efficacy on smooth muscle. The composition-22 obtained when combining these two ingredients at a 1:2 ratio also showed synergism in improving smooth muscle contractility when compared to the efficacy demonstrated by their corresponding individual ingredients as summarized in Table-8.

In another example, Abelmoschus esculentus water extract (A.E-1) and Amorphophallus paeoniifolius water extract (A.P-1) exhibited smooth muscle contractility with EC50 values of 24.62 μg/mL and 20.04 μg/mL, respectively. The composition-23 containing these two extracts at 2:1 ratio showed an EC50 value of 13.88 μg/mL, which is significantly better than the individual ingredients, suggesting a synergistic effect between Abelmoschus esculentus water extract (A.E-1) and Amorphophallus paeoniifolius water extract (A.P-1) in improving contractility efficacy on smooth muscle. The composition-25 obtained when combining these two ingredients at a 1:2 ratio also showed synergism in improving smooth muscle contractility when compared to the efficacy demonstrated by their corresponding individual ingredients as summarized in Table-9.

The inventors of the current application screened these compositions for their inhibition of TNF-α and IL-6 production in comparison with the corresponding individual ingredients.

Significance of TNFα and IL-6 in Gut Inflammation and Immunity

Tumor necrosis factor-α (TNFα) is a pleiotropic, pro-inflammatory cytokine that has multiple biological effects. TNFα is produced by macrophages, monocytes, T cells, B cells upon their stimulation by various factors, including viral infections. TNFα plays a critical role in the pathogenesis of intestinal inflammation. Patients suffering from chronic intestinal inflammation show elevated levels of TNF-α due to elevated numbers of TNFα secreting cells in the intestinal tissue. TNFα also regulates immune responses to the intestinal antigens via controlling multiple aspects of intestinal macrophages and dendritic cell physiology, including their differentiation, migration, maturation, survival, and effector functions. Regulation of TNFα levels via anti-TNFα treatment is an important therapeutic option for patients suffering from conditions related to gut inflation, such as inflammatory bowel disease (IBD).

Interleukin-6 (IL-6) is a multifunctional pro-inflammatory cytokine synthesized by both lymphoid and non-lymphoid cells, which causes inflammation in response to a wide variety of stimuli including infection, stress and trauma. This cytokine is produced by monocytes, macrophages, and epithelial cells and plays a crucial role in the uncontrolled intestinal inflammatory process, which is a main characteristic feature of IBD. The elevated production of IL-6 and its soluble receptor (sIL-6R) by the intestinal macrophages and CD4+T-cells are hallmark features of chronic intestinal inflammation. Several studies have reported that higher circulating IL-6 concentrations in patients with IBD, including Crohn's disease, ulcerative colitis, compared with the healthy controls. Increased level of IL-6 is also correlated with reduced colonic motility that results in gastrointestinal smooth muscle dysfunction.

The TNF-α and IL-6 inhibitory potential of the individual extracts and the compositions were evaluated in human peripheral blood mononuclear cells (PBMC) using human TNFα and IL-6 ELISA immunoassay kits procured from R&D Systems, USA.

TNF-α inhibition: For example, Abelmoschus esculentus water extract (A.E-1) at 0.67 μg/mL and Withania somnifera water extract (W.S-1) at 0.33 μg/mL concentration showed 5.15% and 1.95% reductions of TNF-α, respectively. The composition-2 containing A.E-1 and W.S-1 in the ratio of 2:1 at 1 μg/mL showed a 14.15% reduction of TNF-α, which is significantly higher than the additive effect 7.10% (5.15%+1.95%) calculated from the reduction showed by the corresponding individual ingredients. The compositions-3 & 4 containing these two extracts (A.E-1 and W.S-1) at ratios 1:1 and 1:2 respectively also exhibited synergism when compared to the inhibitions shown by each of their corresponding individual ingredient concentrations, as summarized in Table 10. In an additional example, Abelmoschus esculentus water extract (A.E-1) at 0.67 μg/mL and a blend of Withania somnifera water & 80% aqueous ethanol blend extracts (W.S-2) at 0.33 μg/mL concentration showed 5.15% and 1.72% reductions of TNF-α, respectively. The composition-7 containing A.E-1 and W.S-2 in the ratio of 2:1 at 1 μg/mL showed a 15.39% reduction of TNF-α, which is significantly greater than the additive effect 6.87% (5.15%+1.72%) calculated from the inhibitions showed by the corresponding individual ingredients. The compositions-8 & 9 containing these two extracts (A.E-1 and W.S-2) at ratios 1:1 and 1:2 also exhibited synergism compared to the reductions shown by each of their corresponding individual ingredient concentrations as summarized in Table 10.

IL-6 inhibition: For example, Abelmoschus esculentus water extract (A.E-1) at 0.67 μg/mL and Withania somnifera water extract (W.S-1) at 0.33 μg/mL concentration showed 0.95% and 1.92% reductions of IL-6, respectively. The composition-2 containing A.E-1 and W.S-1 in the ratio of 2:1 at 1 μg/mL showed a 5.62% reduction of IL-6, which is significantly greater than the additive effect 2.87% (0.95%+1.92%) calculated from the inhibitions showed by the corresponding individual ingredients. The compositions-3 & 4 containing these two extracts (A.E-1 and W.S-1) at ratios 1:1 and 1:2 respectively also exhibited synergism when compared to the effect shown by each of their corresponding individual ingredient concentrations, as summarized in Table 11. Similarly, the compositions 7-9 containing Abelmoschus esculentus water extract (A.E-1) and a blend of Withania somnifera water & 80% aqueous ethanol blend extracts (W.S-2) at ratios 2:1, 1:1, and 1:2 respectively also showed significant better reductions 1L-6 levels than their corresponding additive effects, thus established synergism of these compositions (Table-12). Similarly, the compositions 11-13 containing Abelmoschus esculentus ethanol precipitated water extract (A.E-2) and a blend of Withania somnifera water & 80% aqueous ethanol blend extracts (W. S-2) at ratios 2:1, 1:1, and 1:2 respectively also showed higher reduction than their additive effects, thus further establishing the synergistic potential of these compositions (Table 13).

The foregoing thus suggests that the extract(s), or fraction(s) or phytochemicals or mixtures thereof derived from at least two herbs selected from Abelmoschus esculentus, Withania somnifera, and Amorphophallus paeoniifolius; have the tendency to exhibit synergism.

Formulations: The present invention also provides synergistic herbal compositions comprising extract(s), fraction(s), phytochemical(s), or mixtures thereof derived from at least two herbs selected from Abelmoschus esculentus, Withania somnifera, and Amorphophallus paeoniifolius and optionally containing at least one component selected from pharmaceutically or nutraceutically or dietetically acceptable excipients, carriers and diluents.

The synergistic compositions comprising at least two ingredients selected from extract(s), fraction(s), phytochemical(s), or mixtures thereof derived each one from a different herb selected from Abelmoschus esculentus, Withania somnifera and Amorphophallus paeoniifolius and optionally containing at least one component selected from pharmaceutically or nutraceutically or dietically acceptable excipients, carriers and diluents; for improving gut health functions selected from gut brain axis, easing of constipation, bowel movement, improved GI motility and gut immunity, and reducing gut inflammation; wherein the pharmaceutically or nutraceutically or dietically acceptable excipients, carriers and diluents are selected from Monosaccharide's such as glucose, dextrose, fructose, galactose etc.; Disaccharides such as but not limited to sucrose, maltose, lactose, lactulose, trehalose cellobiose, chitobiose etc.; Polycarbohydrates such as starch and modified starch such as sodium starch glycolate, pre-gelatinized starch, soluble starch, and other modified starches; Dextrins that are produced by hydrolysis of starch or glycogen such as yellow dextrin, white dextrin, maltodextrin etc.; Polyhydric alcohols or sugar alcohols such as but not limited to sorbitol, mannitol, inositol, xylitol, isomalt etc.; cellulose based derivatives such as but not limited to microcrystalline cellulose, hydroxy propyl methyl cellulose, hydroxy ethyl cellulose etc.; silicates such as but not limited to neusilin, veegum, talc, colloidal silicon dioxide etc.; metallic stearates such as but not limited to calcium stearate, magnesium stearate, zinc stearate etc.; Organic acids such as citric acid, tartaric acid, malic acid, succinic acid, lactic acid, L-ascorbic acid etc.; Fatty acid esters and esters of poly sorbate, natural gums such as but not limited to acacia, carrageenan, guar gum, xanthan gum etc.; vitamin B group, nicotinamide, calcium pantothenate, amino acids, proteins such as but not limited to casein, gelatin, pectin, agar; organic metal salts such as but not limited to sodium chloride, calcium chloride, dicalcium phosphate, zinc sulphate, zinc chloride etc.; natural pigments, flavors, class I & class II preservatives and aqueous, alcoholic, hydro-alcoholic, organic solutions of above listed ingredients alone or in combination.

Compositions enhance gastro kinetic activity, increases fecal matter and fecal moisture content in male Wistar rats: The disorders associated with gastrointestinal (GI) organ systems include dyspepsia, constipation, gastroesophageal reflux disease, irritable bowel syndrome, etc. In particular, infrequent or slow bowel movement results in constipation, a state where the stool is hard and dry, and difficult to pass. An in vivo experiment was conducted to evaluate the efficacy of the inventive compositions to enhance the gastro-kinetic activity.

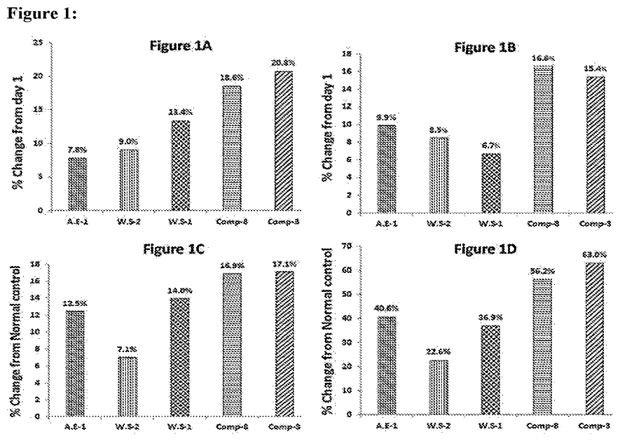

Improving fecal pellet count: The present compositions showed synergistic efficacy in improving the number of fecal pellets in the experimental animals. For example, Abelmoschus esculentus water extract (A.E-1) and a blend of Withania somnifera water and 80% aqueous ethanol extracts (W.S-2) showed efficacy in improving fecal pellet counts by 7.8% and 9.0% respectively when compared to their corresponding values on day 1. The composition-8 containing these two extracts at 1:1 ratio showed 18.6% increase from day 1, which is significantly higher than the individual ingredients, suggesting a synergistic effect between Abelmoschus esculentus water extract (A.E-1) and Withania somnifera water and 80% aqueous ethanol extract (W.S-2) in improving fecal pellet count. Similarly, Abelmoschus esculentus water extract (A.E-1) and Withania somnifera water extract (W.S-1) showed efficacy in improving fecal pellet count by 7.8% and 13.4% from day 1, respectively. The composition-3 containing these two extracts at a 1:1 ratio showed 20.8% increase from day 1, which is significantly higher than the individual ingredients, suggesting a synergistic effect between Abelmoschus esculentus water extract (A.E-1) and Withania somnifera water extract (W.S-1) in improving the fecal pellet count as summarized in FIG. 1A.

Improving fecal moisture content: The present compositions showed synergistic efficacy in improving fecal moisture content activity. For example, Abelmoschus esculentus water extract (A.E-1) and a blend of Withania somnifera water and 80% aqueous ethanol extract (W.S-2) showed efficacy in improving fecal moisture content by 9.9% and 8.5% respectively when compared to their corresponding values on day 1. While, the composition-8 containing these two extracts at 1:1 ratio showed 16.6% increase from day 1, which is significantly higher than the individual ingredients, suggesting a synergistic effect between Abelmoschus esculentus water extract (A.E-1) and a blend of Withania somnifera water and 80% aqueous ethanol extracts (W.S-2) in improving fecal moisture content. Similarly, Abelmoschus esculentus water extract (A.E-1) and Withania somnifera water extract (W.S-1) showed efficacy in improving fecal moisture content from day 1, and the improved values are 9.9% and 6.7%, respectively. While, the composition-3 containing these two extracts at 1:1 ratio showed 15.4% increase, which is significantly higher than the individual ingredients, suggesting a synergistic effect between Abelmoschus esculentus water extract (A.E-1) and Withania somnifera water extract (W.S-1) in improving fecal moisture content as summarized in FIG. 1B.

Improving intestinal transit: The present compositions also showed synergistic efficacy in improving intestinal transit activity. For example, Abelmoschus esculentus water extract (A.E-1) and a blend of Withania somnifera water and 80% aqueous ethanol extracts (W.S-2) showed 12.5% and 7.1% improvements in intestinal transit, respectively compared to the control rats. While, the composition-8 containing these two extracts at 1:1 ratio showed 16.9% increase intestinal trasit from the control rats, which is significantly higher than the individual ingredients, suggesting a synergistic effect between Abelmoschus esculentus water extract (A.E-1) and a blend of Withania somnifera water and 80% aqueous ethanol extract (W.S-2) in improving intestinal transit. Similarly, Abelmoschus esculentus water extract (A.E-1) and Withania somnifera water extract (W.S-1) showed 12.5% and 14.0% improvements in intestinal transit, respectively compared to the control rats. While, the composition-3 containing these two extracts at 1:1 ratio showed 17.1% improvement from the control rats, which is significantly higher than the individual ingredients, suggesting a synergistic effect between Abelmoschus esculentus water extract (A.E-1) and Withania somnifera water extract (W.S-1) in improving intestinal transit as summarized in FIG. 1C.

Improving gastric emptying: The present compositions showed synergistic efficacy in improving gastric emptying activity. For example, Abelmoschus esculentus water extract (A.E-1) and Withania somnifera water and 80% aqueous ethanol extract (W.S-2) showed 40.6% and 22.6% improvement in gastric emptying, respectively compared to the control rats. While, the composition-8 containing these two extracts at a 1:1 ratio showed 56.2% improvement, which is significantly higher than the individual ingredients, suggesting a synergistic effect between Abelmoschus esculentus water extract (A.E-1) and Withania somnifera water and 80% aqueous ethanol extract (W.S-2) in improving gastric emptying. Similarly, Abelmoschus esculentus water extract (A.E-1) and Withania somnifera water extract (W.S-1) showed 40.6% and 36.9% improvements in gastric emptying, respectively, in comparison with the control rats. While, the composition-3 containing these two extracts at a 1:1 ratio showed 63.0% improvement, which is significantly higher than the individual ingredients, suggesting a synergistic effect between Abelmoschus esculentus water extract (A.E-1) and Withania somnifera water extract (W.S-1) in gastric emptying as summarized in FIG. 1D.

The one-way ANOVA revealed that the compositions supplemented rats showed significant improvements in fecal pellet counts and moisture contents compared to the vehicle control rats. The increase in the number of fecal pellets indicates improved bowel movement (peristalsis). This observation is supported by the data obtained from the experiments on gastric emptying and intestinal transit. Also, the compositions supplemented rats showed increased moisture in their fecal matter. This observation suggests an enhanced secretary action of the glands in the gastro-intestinal tract or increased water retention capability of the herbal composition in the intestinal lumen. Maintaining an adequately wet and hydrated milieu in the gastrointestinal lumen is critical for proper digestion, and easy passage of the undigested food substances through the intestinal lumen, and evacuation of the fecal matter. Together, these observations strongly suggest that compositions 3 and 8 enhanced the gastro kinetic activity, improved the intestinal functions such as contractility, glandular secretory activities intestinal transit in the experimental rats.

Corticosterone secreted from the cortical layer of the adrenal glands regulates stress in rodents. Chronic anxiety and depression are integral for neurotransmitter imbalance in the central nervous system (CNS). The “monoaminergic neurotransmitter deficiency” hypothesis suggests that insufficient levels of the monoamine neurotransmitters such as serotonin (5-HT), norepinephrine (NE) are crucial for the symptoms of depression. Serotonin is an essential neurotransmitter in the brain and the enteric (intestinal) nervous system (ENS). The intestinal entero-chromaffin cells are the sensory transducers of intra-luminal stimuli such as pressure. Once serotonin is released from the entero-chromaffin cells, it activates the primary afferent neurons and influences the transmission of information to the central nervous system; this event is essential for the regulation of GI motility. A lack of serotonergic activity results in inadequate gastro-intestinal motility. In the state of chronic stress or depression, the hypothalamic-pituitary-adrenal (HPA) axis is activated due to the reduced monoamines. It stimulates the adrenal cortex to produce adreno-cortical hormones, including glucocorticoids, and in parallel reduces the parasympathetic activity of the vagus nerve of the ENS. Decreased parasympathetic activity reduces the cholinergic activity and results in slow gastro-intestinal movements and insufficient intestinal glandular activities, including reduced digestive enzyme secretions. Together, increased corticosterone and reduced monoamine levels influence GI dysfunction, including slow GI movement and cause constipation.

Thus the key neurohormones that modulate the gastro-intestinal functions, including its motility, were explored. The present observations reveal that the rats supplemented the inventive compositions showed synergistic improvements in the modulation of the key neurohormones such as reduction in corticosterone, and increase in norepinephrine, and serotonin in the serum samples compared to the control rats (Tables 14-16).

Reduction of corticosterone levels: The present compositions showed synergistic efficacy in reducing corticosterone levels in the experimental animals. For example, Abelmoschus esculentus water extract (A.E-1) and Withania somnifera water and 80% aqueous ethanol blend extract (W.S-2) showed 5.73% and 29.16% reduction in corticosterone levels respectively when compared to the control group rats. The composition-8 containing these two extracts at 1:1 ratio showed 46.92% reduction from the control group, which is significantly higher reduction than the corresponding individual ingredients, suggesting a synergistic effect between Abelmoschus esculentus water extract (A.E-1) and Withania somnifera water and 80% aqueous ethanol blend extract (W.S-2) in reducing corticosterone. Similarly, Abelmoschus esculentus water extract (A.E-1) and Withania somnifera water extract (W.S-1) supplementation showed 5.73% and 24.76% reduction in corticosterone levels respectively from control rats, respectively. While, the composition-3 containing these two extracts at 1:1 ratio showed 40.87% reduction in comparison to control animals, which is significantly a higher reduction than the individual ingredients, suggesting a synergistic effect between Abelmoschus esculentus water extract (A.E-1) and Withania somnifera water extract (W.S-1) in decreasing corticosterone levels as summarized in Table-14.

Increase of serotonin levels: The present compositions also showed synergistic efficacy in increasing serotonin levels in the experimental animals. For example, Abelmoschus esculentus water extract (A.E-1) and Withania somnifera water and 80% aqueous ethanol blend extract (W.S-2) supplementation to rats showed 10.98% and 23.48% increase in serotonin levels respectively when compared to control group rats, respectively. The composition-8 containing these two extracts at a 1:1 ratio showed 53.83% increase from the control group, which is significantly higher than the corresponding individual ingredients, suggesting a synergistic effect between Abelmoschus esculentus water extract (A.E-1) and Withania somnifera water and 80% aqueous ethanol blend extract (W.S-2) in increasing of serotonin levels. Similarly, Abelmoschus esculentus water extract (A.E-1) and Withania somnifera water extract (W.S-1) supplemented groups showed 10.98% and 11.77% increase in the serotonin levels respectively in comparison to the control group. The composition-3 containing these two extracts at 1:1 ratio showed 48.25% increase from control animals, which is significantly higher than the individual ingredients, suggesting a synergistic effect between Abelmoschus esculentus water extract (A.E-1) and Withania somnifera water extract (W.S-1) in increasing serotonin levels as summarized in Table-15.

Increase of norepinephrine levels: The present compositions further showed synergistic efficacy in increasing norepinephrine levels in the experimental animals. For example, the rats supplemented with Abelmoschus esculentus water extract (A.E-1) and Withania somnifera water and 80% aqueous ethanol blend extract (W.S-2) showed 33.90% and 16.76% increase in norepinephrine levels respectively when compared to control group rats. The composition-8 containing these two extracts at 1:1 ratio showed 55.52% increase from the control group, which is significantly higher than the corresponding individual ingredients, suggesting a synergistic effect between Abelmoschus esculentus water extract (A.E-1) and Withania somnifera water and 80% aqueous ethanol blend extract (W.S-2) in increasing norepinephrine. Similarly, Abelmoschus esculentus water extract (A.E-1) and Withania somnifera water extract (W.S-1) showed 33.90% and 11.27% increase in norepinephrine levels respectively, from control rats. The composition-3 containing these two extracts at a 1:1 ratio showed 51.41% increase from control animals, which is significantly higher than the individual ingredients, suggesting a synergistic effect between Abelmoschus esculentus water extract (A.E-1) and Withania somnifera water extract (W.S-1) in increasing norepinephrine levels as summarized in Table-16.

Together, from the above data, it is clearly evident that the inventive compositions have the potential synergistic efficacies in improving neurohoromone levels. Thus, these compositions relieve the symptoms of constipation and improve the gut-brain cross-talk by improving the levels of the vital modulatory neurohormones in the gut-brain axis in the experimental rats.

The foregoing thus demonstrates that synergistic herbal compositions comprising extract(s), fraction(s), phytochemical(s) and mixtures thereof derived from at least two herbs selected from Abelmoschus esculentus, Withania somnifera, and Amorphophallus paeoniifolius unexpectedly showed higher contractility efficacy in smooth muscle. These compositions also showed higher efficacy in enhancing gastrokinetic activity, increasing fecal matter and fecal moisture content in male Wistar rats. Further, these compositions also improve the symptoms of constipation and improve the gut-brain cross-talk through improving the levels of the key modulatory neurohormones in the gut-brain-gut axis in the experimental rats. Hence, the said compositions can be useful for improving at least one gut health function selected from gut-brain axis, easing of constipation, bowel movement, GI motility, digestion and gut immunity; and reducing gut inflammation.

Therefore, in an important embodiment, the present invention provides synergistic herbal compositions comprising at least two ingredients selected from extract(s), fraction(s), phytochemical(s), or mixtures thereof derived each one from a different herb selected from Abelmoschus esculentus, Withania somnifera and Amorphophallus paeoniifolius; for improving at least one gut health function selected from gut-brain axis, easing of constipation, bowel movement, improved GI motility, digestion and gut immunity; and reducing gut inflammation.

In an embodiment, the first ingredient is selected from extract(s), fraction(s), phytochemical(s), or mixtures thereof derived from Abelmoschus esculentus, Withania somnifera and Amorphophallus paeoniifolius and the second ingredient is selected from extract(s), fraction(s), phytochemical(s), or mixtures thereof derived from Abelmoschus esculentus, Withania somnifera and Amorphophallus paeoniifolius, wherein, the first and second ingredients are selected from different herbs

In another embodiment, the present invention provides synergistic compositions comprising at least two ingredients selected from extract(s), fraction(s), phytochemical(s), or mixtures thereof derived each one from a different herb selected from Abelmoschus esculentus, Withania somnifera and Amorphophallus paeoniifolius; wherein the concentration of the first ingredient in the composition varies in the range of 10%-90% by weight and the concentration of the second ingredient varies in the range of 90%-10% by weight.

In other exemplary embodiment, the present invention provides synergistic compositions comprising at least two ingredients selected from extract(s), fraction(s), phytochemical(s), or mixtures thereof derived each one from a different herb selected from Abelmoschus esculentus, Withania somnifera and Amorphophallus paeoniifolius and optionally containing at least one component selected from pharmaceutically or nutraceutically or dietetically acceptable excipients, carriers and diluents for improving at least one gut health function selected from gut-brain axis, easing of constipation, bowel movement, improved GI motility, digestion and gut immunity; and reducing gut inflammation.

In another embodiment, the present invention provides synergistic compositions comprising at least two ingredients selected from extract(s), fraction(s), phytochemical(s), or mixtures thereof derived each one from a different herb selected from Abelmoschus esculentus, Withania somnifera and Amorphophallus paeoniifolius and optionally containing at least one component selected from pharmaceutically or nutraceutically or dietically acceptable excipients, carriers and diluents; for improving gut health functions selected from gut brain axis, easing of constipation, bowel movement, improved GI motility, digestion and gut immunity; and reducing gut inflammation; wherein the pharmaceutically or nutraceutically or dietically acceptable excipients, carriers and diluents are selected from Monosaccharide's such as glucose, dextrose, fructose, galactose etc.; Disaccharides such as but not limited to sucrose, maltose, lactose, lactulose, trehalose cellobiose, chitobiose etc.; Polycarbohydrates such as starch and modified starch such as sodium starch glycolate, pre-gelatinized starch, soluble starch, and other modified starches; Dextrins that are produced by hydrolysis of starch or glycogen such as yellow dextrin, white dextrin, maltodextrin etc.; Polyhydric alcohols or sugar alcohols such as but not limited to sorbitol, mannitol, inositol, xylitol, isomalt etc.; cellulose based derivatives such as but not limited to microcrystalline cellulose, hydroxy propyl methyl cellulose, hydroxy ethyl cellulose etc.; silicates such as but not limited to neusilin, veegum, talc, colloidal silicon dioxide etc.; metallic stearates such as but not limited to calcium stearate, magnesium stearate, zinc stearate etc.; Organic acids such as citric acid, tartaric acid, malic acid, succinic acid, lactic acid, L-ascorbic acid etc.; Fatty acid esters and esters of poly sorbate, natural gums such as but not limited to acacia, carrageenan, guar gum, xanthan gum etc.; vitamin B group, nicotinamide, calcium pantothenate, amino acids, proteins such as but not limited to casein, gelatin, pectin, agar; organic metal salts such as but not limited to sodium chloride, calcium chloride, dicalcium phosphate, zinc sulphate, zinc chloride etc.; natural pigments, flavors, class I & class II preservatives and aqueous, alcoholic, hydro-alcoholic, organic solutions of above listed ingredients alone or in combination.

In another embodiment, the invention provides synergistic compositions comprising at least two ingredients selected from extract(s), fraction(s), phytochemical(s), or mixtures thereof derived each one from a different herb selected from Abelmoschus esculentus, Withania somnifera and Amorphophallus paeoniifolius; wherein the extract(s), fraction(s), phytochemical(s) and mixtures thereof is obtained from at least one plant part selected from the group comprising leaves, stems, tender stems, tender twigs, aerial parts, whole fruit, fruit peel rind, seeds, flower heads, root, bark, hardwood or whole plant or mixtures thereof.

In another embodiment, the invention provides synergistic compositions comprising at least two ingredients selected from extract(s), fraction(s), phytochemical(s), or mixtures thereof derived each one from a different herb selected from Abelmoschus esculentus, Withania somnifera and Amorphophallus paeoniifolius; wherein the extract(s), fraction(s), phytochemical(s) or mixtures thereof, are produced using at least one solvent selected from C1-C5 alcohols selected from ethanol, methanol, n-propanol, isopropyl alcohol; ketones selected from acetone, methylisobutyl ketone, chlorinated solvents selected from methylene dichloride and chloroform; water and mixtures thereof, C1-C7 hydrocarbons such as hexane; esters like ethyl acetate and the like and mixtures thereof.

In the other embodiment, the present invention provides synergistic compositions comprising at least two ingredients selected from extract(s), fraction(s), phytochemical(s), or mixtures thereof derived each one from a different herb selected from Abelmoschus esculentus, Withania somnifera and Amorphophallus paeoniifolius; wherein the extract(s), fraction(s), phytochemical(s) or mixtures thereof are standardized to at least one phytochemical reference marker compound or pharmacologically active marker in the extract(s), fraction(s), or mixtures thereof, wherein phytochemical marker compound or group of phytochemical compounds is in the concentration range of 0.01% to 99% by weight of the extract.

In the other embodiment, the present invention provides synergistic compositions comprising at least two ingredients selected from extract(s), fraction(s), phytochemical(s), or mixtures thereof derived each one from a different herb selected from Abelmoschus esculentus, Withania somnifera and Amorphophallus paeoniifolius; wherein said Abelmoschus esculentus fruit extract or fraction is standardized to total polysaccharides; wherein total polysaccharides is in the concentration range of 0.01% to 50% by weight of the composition.

In the other embodiment, the present invention provides synergistic compositions comprising at least two ingredients selected from extract(s), fraction(s), phytochemical(s), or mixtures thereof derived each one from a different herb selected from Abelmoschus esculentus, Withania somnifera and Amorphophallus paeoniifolius; wherein said Withania somnifera root extract or fraction is standardized to total withanolides; wherein total withanolides is in the concentration range of 0.01% to 50% by weight of the composition.

In another embodiment, the present invention provides synergistic compositions comprising at least two ingredients selected from extract(s), fraction(s), phytochemical(s), or mixtures thereof derived each one from a different herb selected from Abelmoschus esculentus, Withania somnifera and Amorphophallus paeoniifolius; wherein the composition(s) are formulated into a dosage form selected from dry powder form, liquid form, beverage, food product, dietary supplement or any suitable form such as tablet, a capsule, a soft chewable or gummy bear.

In another embodiment of the invention, the composition(s) as disclosed above can be formulated into nutritional/dietary supplements that can be contemplated/made into the dosage form of healthy foods or food for specified health uses such as solid food like chocolate or nutritional bars, semi-solid food like cream, jam, or gel or beverage such as refreshing beverage, lactic acid bacteria beverage, drop, candy, chewing gum, gummy candy, yoghurt, ice cream, pudding, soft adzuki bean jelly, jelly, cookie, tea, soft drink, juice, milk, coffee, cereal, snack bar and the like.

In a further embodiment, the present invention provides a method of improving at least one gut health function selected from gut-brain axis, easing of constipation, bowel movement, improved GI motility, digestion and gut immunity; and reducing gut inflammation in a human, wherein the method comprises supplementing the human with an effective dose of a composition comprising at least two ingredients selected from extract(s), fraction(s), phytochemical(s), or mixtures thereof derived each one from a different herb selected from Abelmoschus esculentus, Withania somnifera and Amorphophallus paeoniifolius and optionally containing at least one component selected from pharmaceutically or nutraceutically or dietetically acceptable excipients, carriers and diluents.

In another embodiment, the present invention provides use of a synergistic compositions comprising at least two ingredients selected from extract(s), fraction(s), phytochemical(s), or mixtures thereof derived each one from a different herb selected from Abelmoschus esculentus, Withania somnifera and Amorphophallus paeoniifolius and optionally containing pharmaceutically or nutraceutically or dietetically acceptable carriers/excipients; for improving at least one gut health function selected from gut-brain axis, easing of constipation, bowel movement, improved GI motility, digestion and gut immunity; and reducing gut inflammation in a human.

Those of ordinary skilled in the art will appreciate that changes could be made to the embodiments described above without departing from the broad inventive concept thereof. It is understood, therefore, that this invention is not limited to the particular embodiments or examples disclosed herein, but is intended to cover modifications within the objectives and scope of the present invention as defined in the specification. The presented examples illustrate the invention, but they should not be considered to limit the scope of the invention in any way.

### Example 1: Preparation of Abelmoschus esculentus Water Extract

Abelmoschus esculentus dried whole fruit plant material was pulverized, and the powder (100 g) was added to water (700 mL) at room temperature (rt). The mixture was stirred at 75-80° C. for 3 h and the extract was filtered through celite. The extraction process was repeated with water (2×500 mL) under similar conditions. The combined extracts were filtered and evaporated under reduced pressure to obtain extract concentrate, which was subjected to further drying in a vacuum dryer to give the water extract of Abelmoschus esculentus as a dark brown color powder (A.E-1; 37.0 g).

### Example 2: Preparation of Abelmoschus esculentus Ethanol Precipitated Water Extract

Abelmoschus esculentus dried whole fruit plant material was pulverized, and the powder (100 g) was added to water (700 mL) at rt. The mixture was stirred at 75-80° C. for 3 h and the extract was filtered through celite. The extraction process was repeated with water (2×500 mL) under similar conditions. The combined extracts were filtered and evaporated under reduced pressure to get a concentrated solution. To this concentrate, ethanol (500 mL) was added at rt and stirred for 5 h. The precipitated solution was filtered and washed the powder with ethanol (20 mL). The powder was further subjected to drying in a vacuum dryer to give the ethanol precipitated water extract of Abelmoschus esculentus as an off-white color powder (A.E-2; 17.0 g).

### Example 3: Preparation of Abelmoschus esculentus 50% Aqueous Ethanol Extract

Abelmoschus esculentus dried whole fruit plant material was pulverized and the powder (100 g) was added to 50% aqueous ethanol (800 mL) at rt. The mixture was stirred at 65-70° C. for 3 h and the extract was filtered through celite. The extraction process was repeated with 50% aqueous ethanol (2×600 mL) under similar conditions. The combined extracts were filtered and evaporated under reduced pressure to obtain extract concentrate, which was further subjected to drying in a vacuum dryer to give the 50% aqueous ethanol extract of Abelmoschus esculentus as a dark brown color powder (A.E-3; 29.5 g).

### Example 4: Preparation of Abelmoschus esculentus 50% Aqueous Methanol Extract

Abelmoschus esculentus dried whole fruit plant material was pulverized, and the powder (100 g) was added to 50% aqueous methanol (800 mL) at rt. The mixture was stirred at 65-70° C. for 3 h, and the extract was filtered through celite. The extraction process was repeated with 50% aqueous methanol (2×600 mL) under similar conditions. The combined extracts were filtered and evaporated under reduced pressure to obtain extract concentrate, which was further subjected to drying in a vacuum dryer to give the 50% aqueous methanol extract of Abelmoschus esculentus as a dark brown color powder (A.E-4; 23.8 g).

### Example 5: Preparation of Abelmoschus esculentus 20% Aqueous Acetone Extract

Abelmoschus esculentus dried whole fruit plant material was pulverized, and the powder (100 g) was added to 20% aqueous acetone (800 mL) at rt. The mixture was stirred at 65-70° C. for 3 h, and the extract was filtered through celite. The extraction process was repeated with 20% aqueous acetone (2×600 mL) under similar conditions. The combined extracts were filtered and evaporated under reduced pressure to obtain extract concentrate, which was further subjected to drying in a vacuum dryer to give the 20% aqueous acetone extract of Abelmoschus esculentus as a light brown color powder (A.E-5; 22.5 g).

### Example 6: Standardization of Abelmoschus esculentus Extracts

The various extracts of Abelmoschus esculentus were standardized to total polysaccharides by UV method of analysis, and the results are summarized in Table 1.

TABLE 1
Details of Abelmoschus esculentus extracts

| Example | Extract | Solvent for | Weight of the | |
|---|---|---|---|---|
| # | code | extraction | product | Polysaccharides |
| 1 | A.E-1 | Water extract | 37.0 g | 34% |
| 2 | A.E-2 | Ethanol precipitated | 17.0 g | 49% |
| | | water extract | | |
| 3 | A.E-3 | 50% aq ethanol | 29.5 g | 19.5% |
| 4 | A.E-4 | 50% aq methanol | 23.8 g | 17.7% |
| 5 | A.E-5 | 20% aq acetone | 22.5 g | 12.8% |

### Example 7: Preparation of Withania somnifera Water Extract

Withania somnifera dried root plant raw material was pulverized, and the powder (100 g) was added to water (400 mL) at rt. The mixture was stirred at 70-75° C. for 3 h, and the extract was filtered through celite. The extraction process was repeated with water (2×400 mL) under similar conditions. The combined extracts were filtered and evaporated under reduced pressure to obtain extract concentrate, which was further subjected to drying in a vacuum dryer to give the water extract of Withania somnifera as a brown color powder (W.S-1; 15.8 g).

### Example 8: Preparation of Withania somnifera Water and 80% Aqueous Ethanol Extract

Withania somnifera dried root plant material was pulverized and the powder (100 g) was added to water (300 mL) at rt. The mixture was stirred at 70-75° C. for 3 h and the extract was filtered through celite. The extraction process was repeated with water (2×400 mL) under similar conditions. The combined extracts were filtered and evaporated under reduced pressure to obtain a thick solution (appr 70 mL volume). The spent Withania somnifera root raw material was then extracted with 80% ethanol (400 mL) for 3 h at 70-75° C., and the extract was filtered through celite. The extraction process was repeated with 80% aqueous ethanol (300 mL) under similar conditions. The water extract concentrate and the 80% aqueous ethanol extract solutions were combined, filtered, and evaporated under reduced pressure to obtain a concentrate, which was subjected to further drying in vacuum dryer to give the water and 80% aqueous ethanol extract of Withania somnifera as a brown color powder (W.S-2; 21.25 g).

### Example 9: Preparation of Withania somnifera 80% Aqueous Methanol Extract

Withania somnifera dried root plant raw material was pulverized, and the powder (100 g) was added to 80% aqueous methanol (400 mL) at rt. The mixture was stirred at 65-70° C. for 3 h and the extract was filtered through celite. The extraction process was repeated with 80% aqueous methanol (2×300 mL) under similar conditions. The combined extracts were filtered and evaporated under reduced pressure to obtain extract concentrate, which was further subjected to drying in a vacuum dryer to give the 80% aqueous methanol extract of Withania somnifera as a light brown color powder (W.S-3; 14.5 g).

### Example 10: Preparation of Withania somnifera 80% Aqueous Acetone Extract

Withania somnifera dried root plant raw material was pulverized, and the powder (100 g) was added to 80% aqueous acetone (400 mL) at rt. The mixture was stirred at 65-70° C. for 3 h and the extract was filtered through celite. The extraction process was repeated with 80% aqueous acetone (2×300 mL) under similar conditions. The combined extracts were filtered and evaporated under reduced pressure to obtain extract concentrate, which was further subjected to drying in a vacuum dryer to give the 80% aqueous acetone extract of Withania somnifera as a brown color powder (W.S-4; 9.5 g).

### Example 11: Standardization of Withania somnifera Extracts

The various extracts of Withania somnifera were standardized to total withanolides by the analytical HPLC method (USP method), and the results are summarized in Table 2.

TABLE 2
Details of Withania somnifera extracts

| | | Solvent | Weight | Total |
|---|---|---|---|---|
| Example | Extract | for | of the | withanolides |
| # | code | extraction | product | by HPLC |
| 7 | W.S-1 | Water extract | 15.8 g | 0.35% |
| 8 | W.S-2 | Water and 80% | 21.25 g | 1.2% |
| | | aqueous | | |
| | | ethanol extract | | |
| 9 | W.S-3 | 80% aqueous | 14.5 g | 1.8% |
| | | methanol | | |
| | | extract | | |
| 10 | W.S-4 | 80% aqueous | 9.5 g | 3.1% |
| | | acetone | | |
| | | extract | | |

### Example 12: Preparation of Amorphophallus paeoniifolius Extracts

(a) Water extract: Amorphophallus paeoniifolius dried tuber plant material was pulverized and the powder (100 g) was added to water (700 mL) at rt. The mixture was stirred at ambient temperature for 1 h, and the extract was filtered through celite. The extraction process was repeated with water (2×500 mL) under similar conditions. The combined extracts were filtered and evaporated under reduced pressure to obtain extract concentrate, which was further subjected to drying in a vacuum dryer to give the water extract of Amorphophallus paeoniifolius as a pale brown color powder (A.P-1; 12.8 g).
(b) 50% aqueous ethanol extract: The 50% aqueous ethanol extract (A.P-2; 9.6 g) was obtained from 100 g raw material by adopting a similar procedure using 50% aqueous ethanol as extraction solvent.
(c) Ethanol extract: The ethanol extract (A.P-3; 1.1 g) was obtained from 100 g raw material by adopting a similar procedure using ethanol as extraction solvent.

### Example 13: Preparation of Compositions Containing Abelmoschus esculentus Water Extract (A.E-1) and Withania somnifera Water Extract (W.S-1)

Composition-1 (C-1): The composition-1 was prepared by combining Abelmoschus esculentus water extract (A.E-1) and Withania somnifera water extract (W.S-1) in the ratio of 3:1.

Composition-2 (C-2): The composition-2 was prepared by combining Abelmoschus esculentus water extract (A.E-1) and Withania somnifera water extract (W.S-1) in the ratio of 2:1.

Composition-3 (C-3): The composition-3 was prepared by combining Abelmoschus esculentus water extract (A.E-1) and Withania somnifera water extract (W.S-1) in the ratio of 1:1.

Composition-4 (C-4): The composition-4 was prepared by combining Abelmoschus esculentus water extract (A.E-1) and Withania somnifera water extract (W.S-1) in the ratio of 1:2.

Composition-5 (C-5): The composition-5 was prepared by combining Abelmoschus esculentus water extract (A.E-1) and Withania somnifera water extract (W.S-1) in the ratio of 1:3.

### Example 14: Preparation of Compositions Containing Abelmoschus esculentus Water Extract (A.E-1) and Withania somnifera Water & 80% Aqueous Ethanol Extract (W.S-2)

Composition-6 (C-6): The composition-6 was prepared by combining Abelmoschus esculentus water extract (A.E-1) and Withania somnifera water & 80% aqueous ethanol extract (W.S-2) in the ratio of 3:1.

Composition-7 (C-7): The composition-7 was prepared by combining Abelmoschus esculentus water extract (A.E-1) and Withania somnifera water & 80% aqueous ethanol extract (W.S-2) in the ratio of 2:1.

Composition-8 (C-8): The composition-8 was prepared by combining Abelmoschus esculentus water extract (A.E-1) and Withania somnifera water & 80% aqueous ethanol extract (W.S-2) in the ratio of 1:1.

Composition-9 (C-9): The composition-9 was prepared by combining Abelmoschus esculentus water extract (A.E-1) and Withania somnifera water & 80% aqueous ethanol extract (W.S-2) in the ratio of 1:2.

Composition-10 (C-10): The composition-10 was prepared by combining Abelmoschus esculentus water extract (A.E-1) and Withania somnifera water & 80% aqueous ethanol extract (W.S-2) in the ratio of 1:3.

### Example 15: Preparation of Compositions Containing Abelmoschus esculentus Ethanol Precipitated Water Extract (A.E-2) and Withania somnifera Water & 80% Aqueous Ethanol Extract (W.S-2)

Composition-11 (C-11): The composition-11 was prepared by combining Abelmoschus esculentus ethanol precipitated water extract (A.E-2) and Withania somnifera water & 80% aqueous ethanol extract (W.S-2) in the ratio of 2:1.

Composition-12 (C-12): The composition-12 was prepared by combining Abelmoschus esculentus ethanol precipitated water extract (A.E-2) and Withania somnifera water & 80% aqueous ethanol extract (W.S-2) in the ratio of 1:1.

Composition-13 (C-13): The composition-13 was prepared by combining Abelmoschus esculentus ethanol precipitated water extract (A.E-2) and Withania somnifera water & 80% aqueous ethanol extract (W.S-2) in the ratio of 1:2.

### Example 16: Preparation of Compositions Containing Abelmoschus esculentus 50% Aqueous Ethanol Extract (A.E-3) and Withania somnifera Water & 80% Aqueous Ethanol Extract (W.S-2)

Composition-14 (C-14): The composition-14 was prepared by combining Abelmoschus esculentus 50% aq ethanol extract (A.E-3) and Withania somnifera water & 80% aqueous ethanol extract (W.S-2) in the ratio of 2:1.

Composition-15 (C-15): The composition-15 was prepared by combining Abelmoschus esculentus 50% aq ethanol extract (A.E-3) and Withania somnifera water & 80% aqueous ethanol extract (W.S-2) in the ratio of 1:1.

Composition-16 (C-16): The composition-16 was prepared by combining Abelmoschus esculentus 50% aq ethanol extract (A.E-3) and Withania somnifera water & 80% aqueous ethanol extract (W.S-2) in the ratio of 1:2.

### Example 17: Preparation of Compositions Containing Abelmoschus esculentus 50% Aqueous Methanol Extract (A.E-4) and Withania somnifera 80% Aqueous Acetone Extract (W.S-4)

Composition-17 (C-17): The composition-17 was prepared by combining Abelmoschus esculentus 50% aq methanol extract (A.E-4) and Withania somnifera 80% aqueous acetone extract (W.S-4) in the ratio of 2:1.

Composition-18 (C-18): The composition-18 was prepared by combining Abelmoschus esculentus 50% aq methanol extract (A.E-4) and Withania somnifera 80% aqueous acetone extract (W.S-4) in the ratio of 1:1.

Composition-19 (C-19): The composition-19 was prepared by combining Abelmoschus esculentus 50% aq methanol extract (A.E-4) and Withania somnifera 80% aqueous acetone extract (W.S-4) in the ratio of 1:2.

### Example 18: Preparation of Compositions Containing Abelmoschus esculentus 20% Aqueous Acetone Extract (A.E-5) and Withania somnifera 80% Aqueous Methanol Extract (W.S-3)

Composition-20 (C-20): The composition-20 was prepared by combining Abelmoschus esculentus 20% aq acetone extract (A.E-5) and Withania somnifera 80% aq methanol extract (W.S-3) in the ratio of 2:1.

Composition-21 (C-21): The composition-21 was prepared by combining Abelmoschus esculentus 20% aq acetone extract (A.E-5) and Withania somnifera 80% aq methanol extract (W.S-3) in the ratio of 1:1.

Composition-22 (C-22): The composition-22 was prepared by combining Abelmoschus esculentus 20% aq acetone extract (A.E-5) and Withania somnifera 80% aq methanol extract (W.S-3) in the ratio of 1:2.

### Example 19: Preparation of Compositions Containing Abelmoschus esculentus Water Extract (A.E-1) and Amorphophallus paeoniifolius Water Extract (A.P-1)

Composition-23 (C-23): The composition-23 was prepared by combining Abelmoschus esculentus water extract (A.E-1) and Amorphophallus paeoniifolius water extract (A.P-1) in the ratio of 2:1.

Composition-24 (C-24): The composition-24 was prepared by combining Abelmoschus esculentus water extract (A.E-1) and Amorphophallus paeoniifolius water extract (A.P-1) in the ratio of 1:1.

Composition-25 (C-25): The composition-25 was prepared by combining Abelmoschus esculentus water extract (A.E-1) and Amorphophallus paeoniifolius water extract (A.P-1) in the ratio of 1:2.

### Example 20: Formulation of the Compositions

Composition-26 (C-26): Abelmoschus esculentus water extract (A.E-1, 45 g) was added slowly to 80% aqueous ethanol solution (100 mL) at 70-80° C. under stirring, and after addition, the stirring continued for a further 10-15 min. Withania somnifera water & 80% aqueous ethanol extract (W.S-2, 45 g) was added slowly to the above suspension at the same temperature and after addition, stirring continued for 10-15 min. Then successively added modified starch (2 g) and maltodextrin (6 g) to the suspension and continue stirring for 5-10 min at the same temperature. Ethanol was distilled off at 70-80° C., and distillation continued after the addition of water (100 mL). The resultant slurry was dried in vacuum to give the composition as flakes. These flakes were homogeneously blended with colloidal silicon dioxide (2 g) in a polyethylene cover or any suitable blender and pulverize the material to give the required composition-26.

### Example 21: General Procedure for Smooth Muscle Contractility

Healthy adult male/female Sprague Dawley rats were chosen for the experiment. After euthanasia, a small portion (approximately 1 cm) of ileum from the small intestine was cut and separated and placed in a freshly prepared buffer, i.e., Kreb's solution (pH 7.2-7.4). The isolated ileum was washed thoroughly with buffer to remove the internal remains. One end of the tissue was tied to a tissue holder and the other end to a forced transducer using a thread in an organ bath (Student's organ bath) with stable environmental conditions (Bath volume—30 mL buffer; Temperature—37° C.; Oxygen—continuous supply). Before mounting the tissue to a transducer, the tension load of the transducer was calibrated using 1 g of weight (LabChart 8 Pro, Powerlab 8/30, AD instruments). After calibration, the weight was removed, and the other end of the tissue was attached to the transducer with a tension load adjusted to 0.5 g. After stabilization, a dose-response curve of the standard was obtained using cholinergic receptor agonist (Acetylcholine); with concentrations of 0.1 μM, 0.2 μM, 0.4 μM etc. Then the dose-response curve of the test compounds was obtained with the concentrations of 1 μg, 5 μg, 10 μg, 20 μg, 40 μg, 80 μg, and 100 μg. Data were analyzed by using LabChart calculation module, and EC₅₀ was calculated by plotting a graph between log dose on X-axis and response on Y-axis.

Parameters: Smooth muscle contractility of the test compounds was determined using the dose-response curve, and the results are summarized in tables 3-9.

TABLE 3
Contractility of isolated rat ileum
(Ex-vivo study) data of the compositions
containing Abelmoschus esculentus water
extract (A.E-1) and Withania somnifera
water extract (W.S-1).

| | A.E-1 | W.S-1 | Ratio of | EC50 of the |
|---|---|---|---|---|
| | EC50 | EC50 | A.E-1 + | compositions |
| Comp # | (μg/mL) | (μg/mL) | W.S-1 | (μg/mL) |
| C-1 | 24.62 | 26.80 | 3:1 | 16.00 |
| C-3 | | | 1:1 | 14.08 |
| C-5 | | | 1:3 | 16.58 |

NOTE 1:
Half maximal Effective Concentration (EC50) refers to the concentration of test substance which induces a response halfway between the baseline and maximum after a particular time.
NOTE 2:
Lower the EC50 value is higher the efficacy

TABLE 4
Contractility of isolated rat ileum data
of the compositions containing
Abelmoschus esculentus water extract (A.E-1)
and a blend of Withania somnifera
water and 80% aqueous ethanol extract (W.S-2).

| | A.E-1 | W.S-2 | Ratio of | EC50 of the |
|---|---|---|---|---|
| | EC50 | EC50 | A.E-l + | compositions |
| Comp # | (μg/mL) | (μg/mL) | W.S-2 | (μg/mL) |
| C-6 | 24.62 | 24.79 | 3:1 | 15.98 |
| C-8 | | | 1:1 | 20.83 |
| C-10 | | | 1:3 | 17.77 |

TABLE 5
Contractility of isolated rat ileum
data of the compositions containing
Abelmoschus esculentus ethanol precipitated
water extract (A.E-2) and a blend of
Withania somnifera water and 80%
aqueous ethanol extract (W.S-2).

| | A.E-2 | W.S-2 | Ratio of | EC50 of the |
|---|---|---|---|---|
| | EC50 | EC50 | A.E-2 + | compositions |
| Comp # | (μg/mL) | (μg/mL) | W.S-2 | (μg/mL) |
| C-11 | 19.69 | 22.31 | 2:1 | 14.97 |
| C-13 | | | 1:2 | 12.57 |

TABLE 6
Contractility of isolated rat ileum data
of the compositions containing
Abelmoschus esculentus 50% aq ethanol
extract (A.E-3) and a blend of Withania
somnifera water & 80% aqueous
ethanol extract (W.S-2).

| | A.E-3 | W.S-2 | Ratio of | EC50 of the |
|---|---|---|---|---|
| | EC50 | EC50 | A.E-3 + | compositions |
| Comp # | (μg/mL) | (μg/mL) | W.S-2 | (μg/mL) |
| C-14 | 25.54 | 21.33 | 2:1 | 15.47 |
| C-16 | | | 1:2 | 13.40 |

TABLE 7
Contractility of isolated rat ileum data
of the compositions containing
Abelmoschus esculentus 50% aq methanol
extract (A.E-4) and Withania somnifera
80% aqueous acetone extract (W.S-4).

| | A.E-4 | W.S-4 | Ratio of | EC50 of the |
|---|---|---|---|---|
| | EC50 | EC50 | A.E-4 + | compositions |
| Comp # | (μg/mL) | (μg/mL) | W.S-4 | (μg/mL) |
| C-17 | 22.98 | 21.71 | 2:1 | 16.16 |
| C-19 | | | 1:2 | 17.87 |

TABLE 8
Contractility of isolated rat ileum data
of the compositions containing
Abelmoschus esculentus 20% aq acetone
extract (A.E-5) and Withania somnifera
80% aq methanol extract (W.S-3).

| | A.E-5 | W.S-3 | Ratio of | EC50 of the |
|---|---|---|---|---|
| | EC50 | EC50 | A.E-5 + | compositions |
| Comp # | (μg/mL) | (μg/mL) | W.S-3 | (μg/mL) |
| C-20 | 22.31 | 22.03 | 2:1 | 10.11 |
| C-22 | | | 1:2 | 13.96 |

TABLE 9
Contractility of isolated rat ileum
data of the compositions containing
Abelmoschus esculentus water extract
(A.E-1) and Amorphophallus paeoniifolius
water extract (A.P-1).

| | A.E-1 | A.P-1 | Ratio of | EC50 of the |
|---|---|---|---|---|
| | EC50 | EC50 | A.E-1 + | compositions |
| Comp # | (μg/mL) | (μg/mL) | A.P-1 | (μg/mL) |
| C-23 | 24.62 | 20.04 | 2:1 | 13.88 |
| C-25 | | | 1:2 | 18.76 |

### Example 22: Assays for Tumor Necrosis Factor-α (TNF-α) and Interleukin-6 (IL-6) Inhibition

Human blood was collected from healthy volunteers from a peripheral vein in the presence of 2 mM EDTA. Plasma was separated by centrifugation at 1000 rpm for 10 minutes, and the residual cell pellet was re-suspended in RPMI medium supplemented with 10% FBS and 2 mM EDTA. Thirty milliliters of blood cell suspension was carefully overlaid onto 15 mL of Ficoll/Lymphoprep in a 50 mL falcon tube in the dark, and the tube was centrifuged at 350×g for 30 minutes without using a brake. The buffy coat (interface between medium and Ficoll) containing peripheral blood mononuclear cells (PBMC) was collected carefully in 25 mL of cold 1× phosphate-buffered saline (PBS) and centrifuged at 1200 rpm for 10 minutes. Residual RBCs in the cell pellet were removed by treating with ACK lysis buffer (Gibco Cat #A10492-01) and washed with fresh 1×PBS. PBMC was re-suspended in RPMI medium supplemented with 10% FBS. An equal (0.1×10⁶) number of cells were seeded in each well of a 96-well plate and treated with different concentrations of the test samples. The cells with 0.2% DMSO served as vehicle control. The plate was incubated in a CO₂ incubator at 37° C. for 2 hrs. The cells, except those in the vehicle control culture wells, were treated with bacterial lipopolysaccharide LPS (1000 ng/mL final concentration), and incubated further for 4 hr at 37° C. The culture plate was centrifuged at 270 g for 10 minutes, and the cell-free culture supernatants were used for measuring TNFα and IL-6 concentrations utilizing specific ELISA kits. The human TNFα and IL-6 immunoassay kits were procured from R&D Systems, MN, USA. The assays were performed following the manufacturer's instructions. Absorbance was measured at 450 nm in a microplate reader (Molecular Devices, San Jose, Calif.). Inhibition of cytokines (TNFα or IL-6) was calculated using the following formula.

% reduction of cytokine=[(normalized conc. of cytokine in LPS)−(normalized conc. of cytokine in test sample)]/(normalized conc. of cytokine in LPS)×100

Note: The normalized cytokine (TNFα or IL-6) concentration in the LPS induced or the treated wells was obtained from deducting the values in the test samples from the vehicle control samples.

The results from TNF-α and IL-6 are presented in table-10 and tables 11-13, respectively.

TABLE 10
Inhibition of TNF-α production by the compositions containing
Abelmoschus esculentus water extract (A.E-1) and Withania
somnifera water extract (W.S-1); and Abelmoschus esculentus
water extract (A.E-1) and a blend of Withania somnifera water &
80% aqueous ethanol extract (W.S-2).

| | | | | | | | % Inhibition of | |
|---|---|---|---|---|---|---|---|---|
| | A.E-1 | | W.S-1 | | | | TNF-α | |
| Comp | μg/ | % in- | μg/ | % in- | Ra- | Dose | Additive | Ob- |
| # | mL | crease | mL | crease | tio | μg/mL | (Calculated) | served |
| C-2 | 0.67 | 5.15 | 0.33 | 1.95 | 2:1 | 1 | 7.10 | 14.15 |
| C-3 | 0.50 | 3.84 | 0.50 | 2.96 | 1:1 | 1 | 6.80 | 16.26 |
| C-4 | 0.33 | 2.53 | 0.67 | 3.96 | 1:2 | 1 | 6.49 | 17.32 |
| | A.E-1 | | W.S-2 | | | | | |
| C-7 | 0.67 | 5.15 | 0.33 | 1.72 | 2:1 | 1 | 6.87 | 15.39 |
| C-8 | 0.50 | 3.84 | 0.50 | 2.60 | 1:1 | 1 | 6.44 | 9.93 |
| C-9 | 0.33 | 2.53 | 0.67 | 3.48 | 1:2 | 1 | 6.02 | 16.54 |

TABLE 11
Inhibition of IL-6 production by the compositions containing
Abelmoschus esculentus water extract (A.E-1) and Withania
somnifera water extract (W.S-1).

| | A.E-1 | | W.S-1 | | | | % Inhibition of | |
|---|---|---|---|---|---|---|---|---|
| | | % in- | | % in- | | Dose | IL-6 | |
| Comp | μg/ | hibi- | μg/ | hibi- | Ra- | μg/ | Additive | Ob- |
| # | mL | tion | mL | tion | tio | mL | (Calculated) | served |
| C-2 | 0.67 | 0.95 | 0.33 | 1.92 | 2:1 | 1 | 2.87 | 5.62 |
| C-3 | 0.5 | 0.71 | 0.5 | 2.92 | 1:1 | 1 | 3.63 | 5.81 |
| C-4 | 0.33 | 0.47 | 0.67 | 3.91 | 1:2 | 1 | 4.37 | 6.26 |

TABLE 12
Inhibition of IL-6 production by the compositions containing
Abelmoschus esculentus water extract (A.E-1) and Withania
somnifera water & 80% aqueous ethanol extract (W.S-2).

| | A.E-1 | | W.S-1 | | | | % Inhibition of | |
|---|---|---|---|---|---|---|---|---|
| | | % in- | | % in- | | Dose | IL-6 | |
| Comp | μg/ | hibi- | μg/ | hibi- | Ra- | μg/ | Additive | Ob- |
| # | mL | tion | mL | tion | tio | mL | (Calculated) | served |
| C-7 | 0.67 | 0.95 | 0.33 | 0.86 | 2:1 | 1 | 1.81 | 5.06 |
| C-8 | 0.5 | 0.71 | 0.5 | 1.31 | 1:1 | 1 | 2.02 | 7.63 |
| C-9 | 0.33 | 0.47 | 0.67 | 1.75 | 1:2 | 1 | 2.22 | 5.21 |

TABLE 13
Inhibition of IL-6 production by the compositions containing
Abelmoschus esculentus ethanol precipitated water extract (A.E-2)
and Withania somnifera water & 80% aqueous ethanol extract
(W.S-2).

| | A.E-2 | | W.S-2 | | | | % Inhibition of | |
|---|---|---|---|---|---|---|---|---|
| | | % in- | | % in- | | Dose | IL-6 | |
| Comp | μg/ | hibi- | μg/ | hibi- | Ra- | μg/ | Additive | Ob- |
| # | mL | tion | mL | tion | tio | mL | (Calculated) | served |
| C-11 | 0.67 | 1.78 | 0.33 | 1.54 | 2:1 | 1 | 3.32 | 2.95 |
| C-12 | 0.5 | 1.33 | 0.5 | 2.34 | 1:1 | 1 | 3.67 | 8.05 |
| C-13 | 0.33 | 0.88 | 0.67 | 3.13 | 1:2 | 1 | 4.01 | 3.38 |

### Example 23: In-Vivo Study of Gastrokinetic Activity, Fecal Matter, and Fecal Moisture Content in Male Wistar Rats

After acclimatization, animals were grouped randomly on day 1 into six groups. On days 1 and 6, the experimental animals were kept in metabolic cages; fecal pellets were collected after 24 hours, counted, and moisture contents were estimated. Animals were fasted overnight on day 7; and on day 8; First group is vehicle control; remaining groups were supplemented with Abelmoschus esculentus water extract (A.E-1, 300 mg/Kg), a blend of Withania somnifera water and 80% aqueous ethanol extract (W.S-2, 300 mg/Kg), Withania somnifera water extract (W.S-1, 300 mg/Kg), composition-8 comprising Abelmoschus esculentus water extract (A.E-1) and a blend of Withania somnifera water and 80% aqueous ethanol extract (W.S-2) in 1:1 ratio (300 mg/Kg) and composition-3 comprising Abelmoschus esculentus water extract (A.E-1) and Withania somnifera water extract (W.S-1) in 1:1 ratio (300 mg/Kg) respectively. After 1 hr, all animals received Evans Blue meal via intra-gastric route. All animals were sacrificed 30 minutes post-Evans Blue semi-solid meal; through CO₂ asphyxiation. The stomach and its contents were minced and homogenized within 30 s. The samples were further attenuated to 50 mL with 0.1N NaOH and allowed to stand at room temperature for 1 h. The absorbance of processed samples was measured at a wavelength of 565 nm using a spectrophotometer. The stomach and its contents obtained from a rat of Basal control group sacrificed immediately after gavage of Evans Blue; served as a reference. The length of the small intestine was measured to calculate the intestinal transit of Evans Blue. The distance traveled by the Evans Blue meal in the intestine, from the pylorus to the caecum, was measured using a graduated scale (in centimeter) and expressed as % intestinal transit.

Treatment: Following randomization, Animals in the control group received 0.1% w/v CMC-Na as the vehicle from day 1-8. Animals in other groups received individual test items from day 1-8.

Parameters: Fecal counts, moisture content were measured for normal control and test item treated groups during the in-life phase on day 1 and day 6. After euthanasia, percent gastric emptying and percent intestinal transit were measured. The data is presented in FIG. 1.

Neurohormones: The modulation of key neurohormones such as corticosterone, norepinephrine and serotonin by the present compositions as well as their individual ingredients were evaluated in the serum samples of the experimental animals. The data from the herbal test substance supplemented groups were compared to the control group. The groups represent Abelmoschus esculentus water extract (A.E-1, 300 mg/Kg), Withania somnifera water and 80% aqueous ethanol blend extract (W.S-2, 300 mg/Kg), Withania somnifera water extract (W.S-1, 300 mg/Kg), composition-8 comprising Abelmoschus esculentus water extract (A.E-1) and a blend of Withania somnifera water and 80% aqueous ethanol extracts (W.S-2) in 1:1 ratio (300 mg/Kg) and composition-3 comprising Abelmoschus esculentus water extract (A.E-1) and Withania somnifera water extract (W.S-1) in 1:1 ratio (300 mg/Kg) respectively. The results are presented in Tables 14-16.

TABLE 14
Reduction of Corticosterone

| | Mean | % reduction of |
|---|---|---|
| | Corticosterone | Corticosterone |
| Group | conc. (nM/L) | from control |
| Control | 778.83 | — |
| A.E-1 | 734.22 | 5.73 |
| W.S-2 | 551.70 | 29.16 |
| W.S-1 | 586.03 | 24.76 |
| Comp-8 | 413.41 | 46.92 |
| Comp-3 | 460.52 | 40.87 |

TABLE 15
Increase of Serotonin

| | Mean Serotonin | % increase of |
|---|---|---|
| | Conc. | Serotonin |
| Group | (ng/ml) | from control |
| Control | 263.34 | — |
| A.E-1 | 292.26 | 10.98 |
| W.S-2 | 325.17 | 23.48 |
| W.S-1 | 294.34 | 11.77 |
| Comp-8 | 405.11 | 53.83 |
| Comp-3 | 390.41 | 48.25 |

TABLE 16
Increase of Norepinephrine

| | | % increase of |
|---|---|---|
| | Mean | Norepinephrine |
| | Norepinephrine | from |
| Group | Conc. (ng/ml) | control |
| Control | 5.20 | — |
| A.E-1 | 6.97 | 33.90 |
| W.S-2 | 6.08 | 16.76 |
| W.S-1 | 5.79 | 11.27 |
| Comp-8 | 8.09 | 55.52 |
| Comp-3 | 7.88 | 51.41 |

1-14. (canceled)

15. A composition comprising at least two ingredients selected from the group consisting of:
from 10% to 90% by weight of an extract of Abelmoschus esculentus, based on the combined weight of the at least two ingredients;
from 10% to 90% by weight of an extract of Withania somnifera, based on the combined weight of the at least two ingredients; and
from 10% to 90% by weight of an extract of Amorphophallus paeoniifolius, based on the combined weight of the at least two ingredients;
wherein the composition is effective for improving at least one gut health function selected from gut-brain axis, easing of constipation, bowel movement, gastro-intestinal (GI) motility, digestion and gut immunity; or reducing gut inflammation.

16. The composition as claimed in claim 15, wherein the composition comprises:
from 25% to 75% by weight of the extract of Abelmoschus esculentus, and
from 25 to 75% by weight of the extract of Withania somnifera.

17. The composition as claimed in claim 15, wherein the composition comprises:
from 25% to 75% by weight of the extract of Abelmoschus esculentus, wherein the extract of Abelmoschus esculentus comprises from 12.8% by weight to 49% by weight polysaccharides, and
from 25 to 75% by weight of the extract of Withania somnifera, wherein the extract of Withania somnifera comprises from 0.35% by weight to 3.1% by weight withanolides.

18. The composition as claimed in claim 15, wherein the composition comprises:
from 25% to 75% by weight of the extract of Abelmoschus esculentus, wherein the extract of Abelmoschus esculentus is a fruit extract obtained by extraction with water, an alcohol, a ketone, or a mixture thereof; and
from 25% to 75% by weight of the extract of Withania somnifera, wherein the extract of Withania somnifera is a root extract obtained by extraction with water, an alcohol, a ketone, or a mixture thereof.

19. The composition as claimed in claim 17, further comprising the extract of Amorphophallus paeoniifolius.

20. The composition as claimed in claim 15, wherein the composition comprises:
from 33% to 67% by weight of the extract of Abelmoschus esculentus, and
from 33% to 67% by weight of the extract of Amorphophallus paeoniifolius.

21. The composition as claimed in claim 15, wherein the composition comprises:
from 33% to 67% by weight of the extract of Abelmoschus esculentus, wherein the extract of Abelmoschus esculentus comprises from 12.8% by weight to 49% by weight polysaccharides, and
from 33% to 67% by weight of the extract of Amorphophallus paeoniifolius, wherein the extract of Amorphophallus paeoniifolius is a tuber extract obtained by extraction with water, an alcohol, or a mixture thereof.

22. The composition as claimed in claim 15, wherein the composition comprises:
from 25% to 75% by weight of the extract of Abelmoschus esculentus, wherein the extract of Abelmoschus esculentus is a fruit extract obtained by extraction with water, an alcohol, a ketone, or a mixture thereof; and
from 25% to 75% by weight of the extract of Amorphophallus paeoniifolius, wherein the extract of Amorphophallus paeoniifolius is a tuber extract obtained by extraction with water, an alcohol, or a mixture thereof.

23. The composition as claimed in claim 21, further comprising the extract of Withania somnifera.

24. The composition as claimed in claim 15, wherein the composition comprises:
the extract of Withania somnifera, wherein the extract of Withania somnifera is a root extract obtained by extraction with water, an alcohol, a ketone, or a mixture thereof; and
the extract of Amorphophallus paeoniifolius, wherein the extract of Amorphophallus paeoniifolius is a tuber extract obtained by extraction with water, an alcohol, or a mixture thereof.

25. The composition as claimed in claim 15, wherein:
said extract of Abelmoschus esculentus is standardized to total polysaccharides; wherein a concentration of the total polysaccharides varies in the range of 0.01% to 50% by weight of the composition; and/or
wherein said extract of Withania somnifera is standardized to total withanolides; wherein a concentration of the total withanolides varies range of 0.01% to 50% by weight of the composition.

26. The composition as claimed in claim 15, wherein the compositions further comprises at least one pharmaceutically, nutraceutically, or dietetically acceptable excipient, carrier, or diluent.

27. The composition as claimed in claim 26, wherein the pharmaceutically, nutraceutically, or dietetically acceptable excipient, carrier, or diluent is selected from the group consisting of monosaccharides, disaccharides, polysaccharides, dextrins, polyhydric alcohols, sugar alcohols, cellulose and derivatives thereof, silicates, metallic stearates, organic acids, fatty acid esters, esters of poly sorbate, natural gums, B vitamins, nicotinamide, calcium pantothenate, amino acids, proteins, metal salts, natural pigments, flavors, class I & class II preservatives, and combinations thereof.

28. The composition as claimed in claim 26, wherein the pharmaceutically, nutraceutically or dietetically acceptable excipient, carrier, or diluent is selected from the group consisting of glucose, dextrose, fructose, galactose, sucrose, maltose, lactose, lactulose, trehalose cellobiose, chitobiose, starch, modified starch, sodium starch glycolate, pre-gelatinized starch, soluble starch, yellow dextrin, white dextrin, maltodextrin, sorbitol, mannitol, inositol, xylitol, isomalt, microcrystalline cellulose, hydroxy propyl methyl cellulose, hydroxy ethyl cellulose, magnesium aluminometasilicate, smectite, talc, colloidal silicon dioxide, calcium stearate, magnesium stearate, zinc stearate, citric acid, tartaric acid, malic acid, succinic acid, lactic acid, L-ascorbic acid, fatty acid esters, esters of poly sorbate, acacia, carrageenan, guar gum, xanthan gum, nicotinamide, calcium pantothenate, amino acids, casein, gelatin, pectin, agar, sodium chloride, calcium chloride, dicalcium phosphate, zinc sulphate, zinc chloride, natural pigments, flavors, class I & class II preservatives, and combinations thereof.

29. The composition as claimed in claim 15, wherein the composition is formulated into a dosage form selected from the group consisting of a dry powder form, a liquid form, a beverage, a food product, a dietary supplement, a tablet, a capsule, a soft chewable tablet, and a gummy bear.

30. The composition as claimed in claim 15, wherein the composition is formulated into a food product selected from the group consisting of a chocolate bar, a nutritional bar, a cream, a jam, a gel, a beverage, a lactic acid bacteria beverage, a drop, a candy, a chewing gum, a gummy candy, a yoghurt, an ice cream, a pudding, a soft adzuki bean jelly, a jelly, a cookie, a tea, a soft drink, a juice, milk, coffee, cereal, and a snack bar.

31. The composition as claimed in claim 15, wherein each ingredient is obtained from at least one plant part selected from the group consisting of leaves, stems, twigs, aerial parts, a whole fruit, a fruit peel rind, seeds, flower heads, a root, bark, hardwood, a whole plant, and mixtures thereof.

32. The composition as claimed in claim 15, wherein each ingredient is obtained using a solvent selected from the group consisting of C1-C5 alcohols, ketones, chlorinated solvents, water, C1-C7 hydrocarbons, esters, and mixtures thereof.

33. The synergistic composition as claimed in claim 15, wherein the extract(s), fraction(s), phytochemical(s) or mixtures thereof; are produced using at least one solvent selected from the group consisting of ethanol, methanol, n-propanol, isopropyl alcohol, acetone, methyl isobutyl ketone, methylene dichloride, chloroform, water, hexane, ethyl acetate, and mixtures thereof.

34. A method of improving at least one gut health function selected from gut-brain axis, easing of constipation, bowel movement, GI motility, digestion and gut immunity; and reducing gut inflammation in a human, wherein the method comprises supplementing the human with an effective dose of a composition according to claim 15.
